(19) United States
(12) Patent Application Publication
(10) Pub. No.: US 2003/0236457 A1
(43) Pub. Date: Dec. 25, 2003

(54) Method of endovascular brain mapping

(76) Inventors: Robert A. Mericle, Gainesville, FL (US); Christopher D. Batich, Gainesville, FL (US); Courtney S. Watkins, Gainesville, FL (US); Matthew V. Burry, Gainesville, FL (US); O. Erich Richter, Gainesville, FL (US); Swadeshmukul Santra, Gainesville, FL (US)

Correspondence Address: Dennis P. Clarke, Miles & Stockbridge P.C., Suite 500, 1751 Pinnacle Drive, McLean, VA, 22102-3833, US

(21) Appl. No.: 10/421,950

(22) Filed: Apr. 24, 2003

Related U.S. Application Data
(60) Provisional application No. 60/374,815, filed on Apr. 24, 2002.

(51) Int. Cl. A61B 5/05

(52) U.S. Cl. 600/420

(57) ABSTRACT

Disclosed are the following strategies for endovascularly mapping the brain with a chemical agent capable of staining the preselected region of the brain to a color visibly contrasting with non-stained portions of the brain and of passing through the blood-brain barrier to the preselected region of the brain: 1) Passive transport: (a) pro-drug, (b) Modification of mapping agent to mimic molecules that readily cross the BBB (e.g., amino acid, glucose, etc.); 2) Active transport; 3) Receptor-mediated transport (e.g., antibody mediated); 4) Blood brain barrier (BBB) manipulation; 5) Emulsification of agent (such as carotene, organic dye, etc.) to stain capillary endothelium; 6) Embolization of brain capillaries; 7) Grafted-nanoparticle systems for brain delivery of mapping agent.

### BACKGROUND OF THE INVENTION

1. Field of the Invention

The present invention relates to a novel brain mapping system especially adapted for the enhancement of Craniotomy Guidance.

2. Description of the Prior Art

Brain mapping is the attempt to specify in as much detail as possible the localization of function in the human brain (Savoy R L: History and future directions of human brain mapping and functional neuroimaging. Acta sychologica 07:9-42, 2001). A variety of brain-mapping techniques exist, ranging from the historic lesion studies to functional magnetic resonance imaging (fMRI) (Llinás, R R: Mapping Brain Terrain. Neurobiology of Disease 7 499-500, 2000; Orison W W, Lewine J P, Sanders J A, Hartshorne M F: Functional Brain Imaging. St. Louis: Mosby, 1995.

The oldest and simplest form of brain mapping is lesion studies (Corkin 5, Amaral D G, Johnson K A, Hyman B T: H.M's medial temporal lobe lesion: findings from MRI. Journal of Neuroscience 17:3964-3979, 1997; Kolb B, Whishaw I Q: Fundamentals of human neuropsychology (₄th ed.). New York: Freeman, 1996; Tootell R B H, Iale A M, Sereno M I, Malach R: New images from human visual cortex. Trends in Neuroscience 19(11):481-489, 1996). The anatomic basis of neurological function has been inferred from the correlation of clinical findings with anatomical studies, whether by autopsy or radiology, in patients with known discrete brain lesions. This is the method by which the knowledge of localization was long advanced, but with marked limitations. Lesions are not controlled and often involve multiple areas with multiple functions. Gray matter subserving one function often overlies white matter carrying fibers subserving an unrelated function. Nevertheless, our basic understanding of neuroanatomy is largely based on these historic studies, and forms the basis for the crude form of preoperative brain mapping currently employed for many neurosurgical procedures the expectation of the neurosurgeon that a given resection has a certain probability of producing a given neurologic deficit.

Starting with Penfield and his associates, the clinical utility of brain mapping for operative planning led to the practice of intraoperative stimulation in awake patients (Penfield, supra; Penfield W, Jasper H: Epilepsy and the functional anatomy of the human brain. Boston, Mass.: Little, Brown and Company, 1954). This was most useful for mapping the motor or language cortex, but found application in other areas as well. These studies, in addition to the lesion studies discussed above, provided much of the current understanding of basic neuroanatomy. The disadvantages of this approach, however, are clear. The increased operative time and associated risk of infection, the broad cortical exposure required, and the inconvenience of completely awakening the patient during open brain surgery while adequately controlling pain all limit the use of these techniques. The process is also painstakingly slow, testing point by point accompanied by questioning of examination of the patient. Additionally, the functions tested are limited to those that can be tested at the bedside in a patient that is physically unable to move the head. Obviously, gait functions or postural stability could not be assessed in this way. Similarly, detailed neuropsychological testing is generally impractical in such a setting.

A more modern development of this technique, primarily employed in preoperative planning of epilepsy surgery, is the use of intracortical depth electrodes and arrays of subdural electrodes that are surgically implanted and then used to collect data passively for a period of time before the definitive neurosurgical procedure (Puce A, Constable R T, Luby M L, McCarthy G, Nobre A C, Spencer D D, Gore J C, Allison T: Functional magnetic resonance imaging of sensory and motor cortex: comparison with electrophysiological localization. Journal of Neurosurgery 832(2):262-270, 1995.). Similarly, many neurosurgeons use microelectrode recording and stimulation to precisely localize deep nuclei (Halgren E, Walter R D, Cherlow D G, Crandall P H: Mental phenomena evoked by electrical stimulation of the human hippocampal formation and amygdala. Brain 101:83-1 17, 1978; Vignal J P, Chauvel P, Halgren E: Localized face processing by the human prefrontal cortex: imulation-evoked hallucinations of faces. Cognitive Neuropsychology, 17:281-291, 2000.). The obvious disadvantage of these approaches is their invasive nature that requires more than one major open brain operation. This includes the accompanying risks of hemorrhage and neurological deficit secondary to direct tissue trauma.

Two modern techniques offer the ability to perform similar tests non-invasively by using electromagnetic fields to induce changes within the cortex that stimulate the tissue or that behave as a temporary lesion. In transcranial electrical stimulation (TES), electrodes are affixed to the scalp and current is passed between them to stimulate underlying cortex. As the procedure is uncomfortable, this technique is seldom used. Transcranial magnetic stimulation (TMS) typically causes no direct unpleasant effects other than noise, and is thus more frequently employed. In TMS, large coils are used to generate rapidly changing magnetic fields within the brain, inducing localized currents that can either stimulate the cortex in that location or temporarily inactivate that cortex (George M S, Lisanby S H, Sackeim H A: Transcranial magnetic stimulation: applications in neuropsychiatry. Archives of General Psychiatry 56:300-311, 199; Pascual-Leone A, Bartres-Faz D, Keenan J P: Transcranial magnetic stimulation: studying the brain-behavior relationship by induction of ‘virtual lesions’, Philosophical Transactions of the Royal Society of London B 354:1229-1238, 1999; Pascual-Leone A, Meador K J: Is transcranial magnetic stimulation coming of age? Journal of Clinical Neurophysiology 15(4):285-287, 1998; Pascual-Leone A, Walsh V, Rothwell J: Transcranial magnetic stimulation in cognitive neuroscience-virtual lesion, chronometry, and functional connectivity, Current Opinion in Neurobiology 10:232-237, 2000; Stewart L, Ellison A, Walsh V, Cowey A: The role of Transcranial Magnetic Stimulation (TMS) in studies of vision, attention and cognition. Acta Psychologica 107:275-291, 2001; Walsh V, Cowey A: Magnetic stimulation studies of visual cognition. Trends in Cognitive Neuroscience 2(3):103-1 10, 1998). The limitations of this technique are its very limited spatial resolution, imprecise location, and the tendency of repetitive stimulation to induce seizures.

Passive transcranial modalities have been employed as well. The most widespread is electroencephalography (EEG), more commonly used for the evaluation of seizure disorders (Gevins A: Electrophysiological imaging of brain function, in Toga A W, Mazziotta J C (Eds.): Brain mapping: the methods. San Diego: Academic Press, 1996, 259-276; Lewine J D, Orrison W W J: Clinical electroencephalography and event-related potentials, in Ctrrison W W J, Lewine J D, Sanders J A, Hartshorne M F (eds): Functional brain imaging 327-368. Boston, Mass.: Mosby-Year Book, 1995). It shares with its magnetic counterpart, magnetoencephalography (MEG) (Lewine J D, Orrison W W J: Magnetoencephalography and magnetic source imaging, in Orison W W J, Lewine J D, Sanders J A, Hartshorne M F (Eds.): Functional brain imaging. Boston: Mosby-Year Book, 1995, 369-4 18; Lounasmaa O V, Hämäläinen M, Han R, Salmelin R: Information processing in the human brain: magnetoencephalographic approach. Proceedings of the National Academy of Science, USA 93:8809-8815, 1996), good the benefit of excellent temporal resolution, but is severely limited in its application to brain mapping by very poor spatial resolution.

Finally, the autoregulation of the brain in increasing local blood flow according to local metabolic activity (neuronal firing) allows for several brain mapping techniques (Raichle M E: Behind the scenes in functional brains imaging: a historical and physiological perspective. Proceedings of the National Academy of Sciences, USA, 95:765-772, 1998.), including most forms of functional Magnetic Resonance Imaging (fMRI), positron emission tomography (PET) scanning, or even optical measurement. These are very powerful techniques, but suffer from the same weakness that all noninvasive strategies do when it comes to preoperative planning, namely, inadequate spatial resolution correlated to the view of the surgeon at craniotomy. Also, while they accurately identify the tissue that is active during any given task, it is difficult without a lesion type study to discern incidentally active tissue from tissue indispensable for neurologic functioning. Generally, the question answered is “What tissue is active during task A?” rather than the question more important to the surgeon, “What are all of the functions that will be impaired by the loss of this tissue, and to what degree?”

Each of these known techniques has various strengths and weaknesses that are useful for certain specific aspects of research in cognitive neuroscience. However, the primary use of brain mapping in neurosurgery is the determination of the presence or absence of function in a potential site of brain resection. Regardless of the indication for neurosurgical resection, it is always desirable to avoid neurological deficits, and so it is desirable to attempt to clarify the function of the tissue before resection. Most existing brain mapping techniques have some characteristics that are favorable for preoperative planning. However, none of the existing brain mapping techniques allows precise, direct, real-time visualization of brain parenchyma that can be resected with assurance of no new post-operative neurological deficit, i.e., “silent” brain parenchyma.

The ideal clinical brain mapping technique, however, allows precise definition of the postoperative deficits to be expected with removal of a given portion of brain tissue, coupled with the ability to subsequently precisely identify that tissue at the time of craniotomy.

### SUMMARY OF THE INVENTION

The present invention relates to a new form of preoperative clinical brain mapping using an endovascular approach that will promote improved neurological outcomes after brain resection. This procedure is not intended for experimental cognitive neuroscience, where the ability to compare results across multiple subjects and serially in time is important. Rather, this technique is especially designed for individualizing the unique brain function map of each patient who requires brain resection to improve his/her neurosurgical outcome.

One embodiment of the invention relates to a method for diagnostically imaging a preselected region in a warm-blooded animal having a brain situated inside a blood-brain barrier having an ambient permeability level, the method comprising the steps:

1) endovascularly administering a first chemical agent to the animal at a location proximal to the preselected region outside the blood-brain barrier, where the first chemical agent causes local brain anesthesia to the portion of the brain which is distal to the position of the microcatheter (targeted brain). This first chemical agent is usually a very short acting barbiturate medication such as Sodium Methohexital (Brevital®), or amobarbital (Amytal®)). This constitutes a “Superselective distal Wada test” (van Emde Boas W: Juhn A. Wada and the sodium amytal test in the first (and last?) 50 years J Hist Neurosci 1999 December; 8(3):286-92).

2) Performing detailed neurological, cognitive, and possibly psychological bedside testing to determine the presence or absence of any new deficit.

3) endovascularly administering a second chemical or physical agent through the same microcatheter which has remained in the exact same position after administration of the first chemical agent proximal to the preselected targeted brain tissue following the administration of the first chemical agent, the second chemical or physical agent being capable of staining the preselected region of the brain to a color visibly contrasting with non-stained portions of the brain, possibly passing through the animal's blood-brain barrier to the preselected region of the brain when the blood-brain barrier is at the elevated permeability level but not at the ambient permeability level. Alternatively, the second chemical agent could be attached to the blood-brain barrier at the level of the brain capillary endothelial cells, or other location within the pre-selected targeted portion of brain that was previously tested with the selective distal Wada test using the first chemical agent as described above.

A second embodiment of the invention concerns an improved method involving surgery on the brain of a warm-blooded mammal, the improvement comprising diagnostically imaging a preselected region in a warm-blooded animal having a brain situated inside a blood-brain barrier having an ambient permeability level, the imaging comprising the steps:

endovascularly administering a first chemical agent to the animal at a location proximal to the preselected region outside the blood-brain barrier, where the first chemical agent is capable of increasing the permeability of the blood-brain barrier from the ambient level to an elevated permeability level;

endovascularly administering a second chemical agent to the animal at a location proximal to the preselected region outside the blood-brain barrier following the administration of the first chemical agent, the second chemical agent being capable of staining the preselected region of the brain to a color visibly contrasting with non-stained portions of the brain and of passing through the animal's blood-brain barrier to the preselected region of the brain when the blood-brain barrier is at the elevated permeability level but not at the ambient permeability level.

Additional embodiments of the invention relate to the following strategies for endovascularly mapping the brain: 1) Passive transport: (a) pro-drug, (b) Modification of mapping agent to mimic molecules that readily cross the BBB (e.g., amino acid, glucose, etc.); 2) Active transport; 3) Receptor-mediated transport (e.g., antibody mediated); 4) Blood brain barrier (BBB) manipulation; 5) Emulsification of agent (such as β-carotene, organic dye, etc.) to stain capillary endothelium; 6) Embolization of brain capillaries; 7) Grafted-nanoparticle systems for brain delivery of mapping agent.

Still further embodiments of the invention relate to kits for endovascularly mapping the brain and their use wherein the kits comprise, as a first component, a chemical agent being capable of staining a preselected region of the brain to a color visibly contrasting with non-stained portions of the brain and, as further components, agents for enhancing transport to the preselected regions of the brain of the chemical agent.

Other embodiments of the invention relate to articles of manufacture and their use wherein the comprises packaging material and a pharmaceutical agent contained within the packaging material, wherein the pharmaceutical agent is effective for the endovascular mapping of the brain, and wherein the packaging material comprises a label which indicates that the pharmaceutical agent can be used for the endovascular mapping of the brain.

### BRIEF DESCRIPTION OF THE DRAWINGS

FIG. 1 depicts a fluorescein mapped rat brain; (a) dorsal view and (b) coronal section.

FIG. 2 depicts a FD & C green #3 mapped rat brain; (a) dorsal view, (b) ventral view and (c) coronal section.

FIG. 3 sets forth the chemical structures of Iohexol (Omnipaque™) (a), Methohexital Sodium; 2,4,6 (1H, 3H, 5H) Pyrimidinetrione, 1-methyl-5-(1-methyl-2-pentynyl)-5-(2-propenyl)-, (±)-, monosodium salt (Brevital®) (b), γ-cyclodextrin (c) and doxorubicin (d).

### DETAILED DESCRIPTION OF THE INVENTION

The novel methods of the invention enable those skilled in the art, e.g., to safely define margins of resection at craniotomy, along with identifying any expected neurological deficits associated with the resection.

There currently exists no system or procedure for the endovascular mapping of the brain. The Wada test, which is performed by infusing a short acting barbiturate, Brevital® (Jones Pharma Inc., St. Louis, Mo.) or sodium amytal, into the internal carotid artery in an awake patient, followed by detailed bedside neurological and cognitive testing, is a routine endovascular procedure that is performed to determine localization of dominant brain function such as language prior to major epilepsy surgery. The test is well known to most interventional neuroradiologists and endovascular neurosurgeons.

Any transient neurological or cognitive deficits that are detected are attributable to the portion of brain parenchyma that was infused with the short-acting barbiturate. The temporary deficits which occur because of Brevital® resolve back to baseline within several minutes; whereas the temporary deficits secondary to sodium amytal may last significantly longer. Because of recent improvements in endovascular techniques and microcatheter technology, it is now routine to catheterize distal branches of the anterior, middle, and posterior cerebral arteries. It is therefore possible to perform a superselective Wada test with transcatheter infusion of Brevital® at distal selective targeted brain parenchyma. This type of superselective Wada testing is often performed prior to endovascular embolization of intracranial pial arteriovenous malformations. Detailed neurological and cognitive examination is performed before and after the transcatheter super-selective transient anesthesia, and this would definitively determine if the target brain parenchyma is “silent” or “eloquent”. If the target brain was proven to be “silent”, an appropriate mapping agent could then be infiltrated into the target brain, so that the “silent” brain is clearly visualized during a subsequent elective craniotomy. The neurosurgeon would then be certain that no deficit would occur after resection of any brain that was mapped as silent. This could be useful for epilepsy surgery, malignant glioma surgery, arteriovenous malformation (AVM) surgery, functional neurosurgical resections, and other possible applications.

With the advent of modern endovascular techniques and the explosion in catheter technology, it is now relatively routine to catheterize and selectively inject 3^{th} and 4^{th} order intracranial vessels. It is possible, therefore, to perform superselective anesthesia endovascularly, essentially inducing a transient reversible neurologic lesion in selected and targetedsections in the brain. Since angiography is performed with the patient awake, the patient may be examined for any new deficit while the anesthetic is in effect. This could be extended beyond basic neurologic examination to include even detailed neuropsychological testing. The clinical usefulness of this ability for planning surgical resections has to date been limited by the difficulty of correlating the findings at endovascular testing with the view of the surgeon at craniotomy.

The present invention enables the marking of the vascular territory being studied with a visible color, thereby enabling the neurosurgeon to precisely identify the tested territory. The surgeon would then be certain that no neurological deficit would result from of the resection of brain that was so marked or “mapped.” This would result in vast improvements in epilepsy surgery, brain tumor surgery, AVM surgery, and the like.

Several dyes are available that are widely used in foods and medications that are minimally toxic. Prior studies have shown that many dyes can cross the blood brain barrier and stain the brain parenchyma after disruption of the blood brain barrier with an osmotic agent (Robinson P J, Rapoport S I: Size selectivity of blood-brain barrier permeability at various times after osmotic opening. Am J Physiol 253:R459-R466, 1987; Suzuki M, Iwasaki Y, Yamamoto T, Konno H, Kudo H: Sequelae of the osmotic blood-brain barrier opening in rats. J Neurosurg 69:42 1-428, 1988; Ziylan Y Z, Robinson P J, Rapoport S I: Differential Blood-Brain Barrier Permeabilities to [¹⁴C]Sucrose and [³H]Inulin after Osmotic Opening in the Rat). Fluorescein is a dye with minimal toxicity widely used in clinical practice, particularly in ophthalmological applications (Albert D M and Jakobiec F A: Principles and Practice of Ophthalmology Clinical Practice. Philadelphia, Pa.: W B Saunders Co, 1994. Fluorescein angiography is an established method of retinal evaluation. Accordingly, the first experiments that led to the present invention explored the use of selective blood-brain barrier disruption followed by fluorescein infusion.

The ideal endovascular brain mapping agent should possess all of the following characteristics: 1) Must be nontoxic systemically; 2) Must be clearly visualized, either directly or indirectly with the assistance of accessory devices (ultraviolet illumination, fluorescent detection, etc.; 3) Must be radiographically opaque for fluoroscopic visualization; 4) Must be capable of either crossing the blood-brain barrier (BBB) either with or without temporary BBB manipulation techniques, or attaching to the capillaries within the selected targeted brain.

The seven major approaches for accomplishing the goal of preoperative endovascular brain mapping include: 1) Passive transport: (a) pro-drug, (b) Modification of mapping agent to mimic molecules that readily cross the BBB (e.g., amino acid, glucose, etc.); 2) Active transport; 3) Receptor-mediated transport (e.g., antibody mediated); 4) Blood brain barrier (BBB) manipulation; 5) Emulsification of agent (such as β-carotene, organic dye, etc.) to stain capillary endothelium; 6) Embolization of brain capillaries; 7) Grafted-nanoparticle systems for brain delivery of mapping agent.

Modifications to the Zeiss operating microscope to allow clear visualization of fluorescein stained brain territories have been described (Kuroiwa T, Kajimoto Y, Ohta T: Development of a Fluorescein operative microscope for use during malignant glioma surgery. A technical note and preliminary report, Surg Neurol 50:41-9, 1998.). However, the advantage in the simplicity of a visible spectrum dye is obvious. Therefore, selective blood brain barrier disruption followed by infusion of an aqueous suspension of FD&C Green #3 was attempted.

Once a given distribution of tissue has been tested and chosen for removal, preoperative embolization of that territory would be helpful to reduce operative bleeding during resection. It was theorized that if an appropriately colored embolic agent were used, it could contribute to the marking of the territory intended for resection. It was therefore decided to test the injection of tantalum powder, a black embolic agent that is radiopaque.

The first step of the endovascular brain mapping procedure is to perform superselective microcatheterization of the target artery feeding the target brain parenchyma in an awake patient in the endovascular suite. Secondly, the target brain parenchyma is infused with methohexital sodium (Brevital®) and performance of detailed neurological examination. If this provocative endovascular testing shows no change in the neurological or cognitive function, then this distribution is defined as “silent” brain parenchyma and is suitable for mapping. An appropriate mapping agent is then infused into the exact same distribution that will visually stain the “silent” brain parenchyma so that when craniotomy is performed, a clear, real-time margin is visualized and will allow the neurosurgeon to resect only the portion that has been proven to be “silent”. If critical neurological function is found in the target brain parenchyma, then the microcatheter must be moved to another adjacent territory. This procedure is repeated at any distribution in the brain that may require surgical resection at a later date. At the conclusion of the preoperative endovascular brain mapping procedure, all silent brain parenchyma in the region of the target parenchyma will be “mapped” or visually marked with clear margins, in conjunction with embolization, if necessary. The final step in the procedure is to perform a previously planned craniotomy and resection of the target lesion. The resection will be guided by the clearly visible mapped silent brain. Therefore, it should be absolutely certain that no neurological or cognitive deficit would occur after the brain resection. This will eliminate the need for other more cumbersome, tedious, painful, and less accurate functional brain mapping procedures such as awake cortical stimulation, electrocorticography with prolonged inpatient monitoring, and stereotactic image-guided surgery.

The feasibility of brain-mapping is illustrated by the following non-limiting examples carried out in animal models. It will be understood by those skilled in the art that before any of the possible mapping agents is attempted in a human, it must first be confirmed to show good visual clarity, and sharp margins between the mapped brain and the non-mapped brain in an animal model.

Generally, Sprague Dawley rats were anesthetized and vascular access obtained by ventral cervical cutdown. A catheter was introduced into the common carotid artery proximal to the ICA. All other branches were clamped temporarily with aneurysm clips, permitting flow to the ICA alone. The blood brain barrier was disrupted with a solution of arabinose. An aqueous solution of Fluorescite® (fluorescein-Alcon Lab) or FD&C Green #3 with Omnipaque® (tantalum powder-Hycomed) contrast agent was infused. Tantalum powder was also tested without blood brain barrier disruption. The brains were harvested for examination of the quality of staining.

### EXAMPLE 1

Female Sprague Dawley rats were anesthetized with a mixture of Ketamine and Xylazine (87 mg/ml/13 mg/ml, 0.1 ml/100 g body weight). Using a carotid cutdown method, the CCA was exposed and a catheter was introduced into the lumen proximal to the ICA. A ligature secured the catheter within the vessel. The external carotid artery and occipital artery were clamped, preserving flow to the ICA. We followed a previously described method for osmotic disruption of the blood brain barrier using a hypertonic solution of arabinose (2.7 g of arabinose was dissolved in 10 ml of sterile water under gentle warming, and the solution was filtered to remove any undissolved crystals. 3.6 cc of this solution was infused over 30 seconds. After a delay of six minutes, we infused a 10% solution of fluorescein mixed with an equal amount of Omnipaque® (Hycomed) contrast agent under fluoroscopic guidance. The animal was then euthanized and the brain harvested.

### EXAMPLE 2

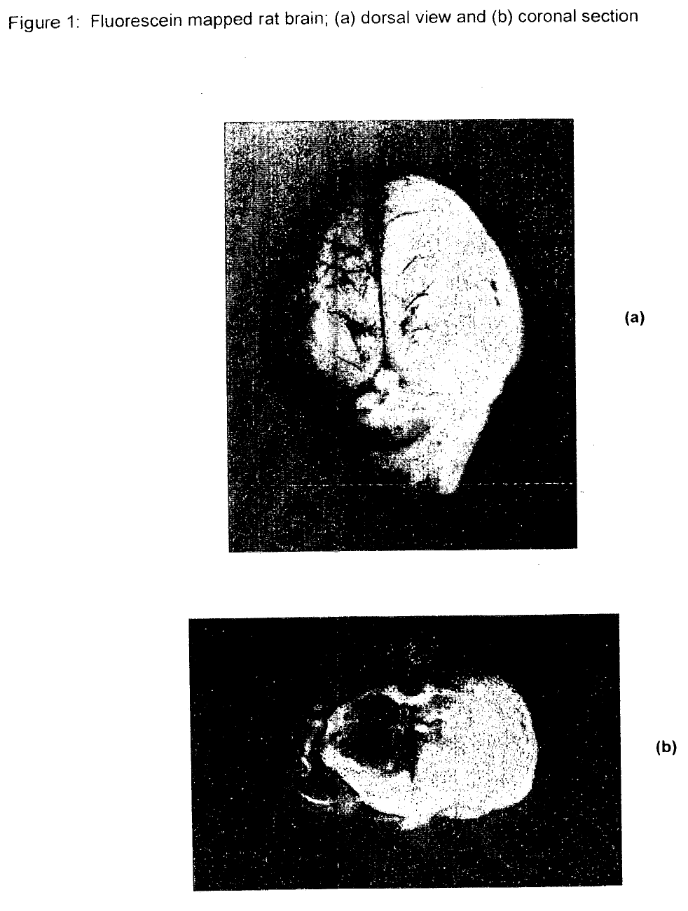

The same procedure was repeated in another rat with an aqueous suspension of FD & C green #3 (FIG. 1) mixed with Omnipaque®. To test the use of tantalum powder, the same procedure described above was performed without disruption of the blood brain barrier. A small amount of tantalum powder suspension was then injected under fluoroscopic visualization until the right hemispheric distribution was opacified.

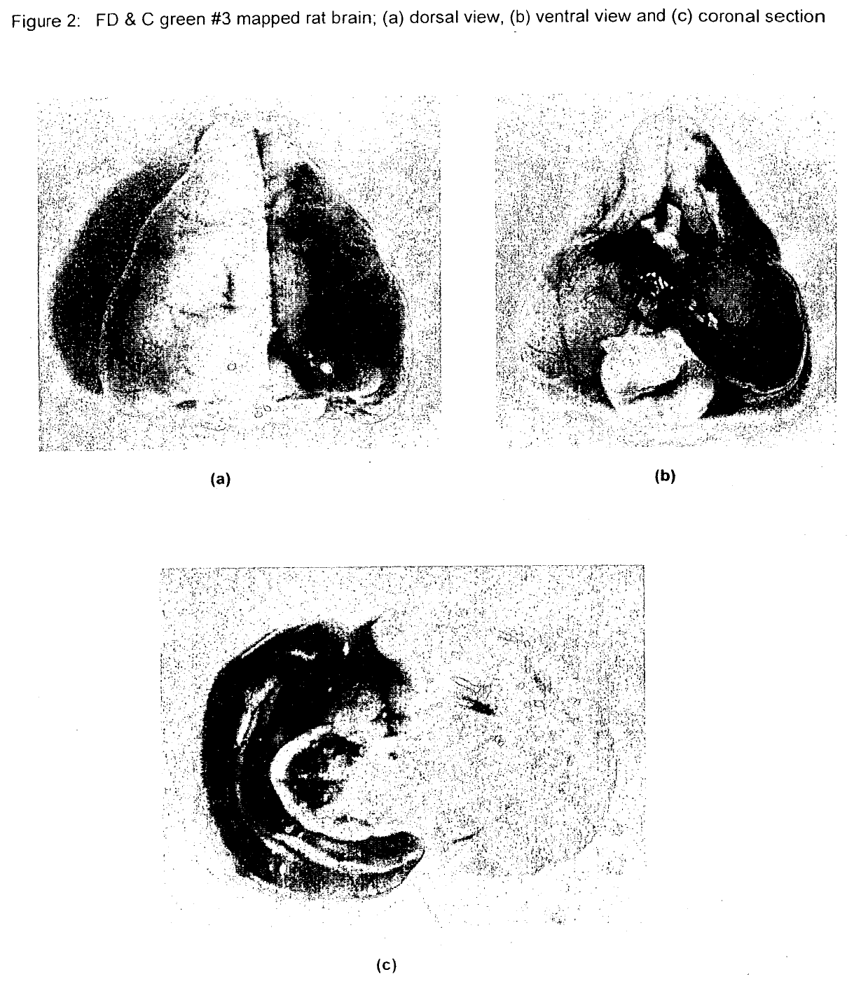

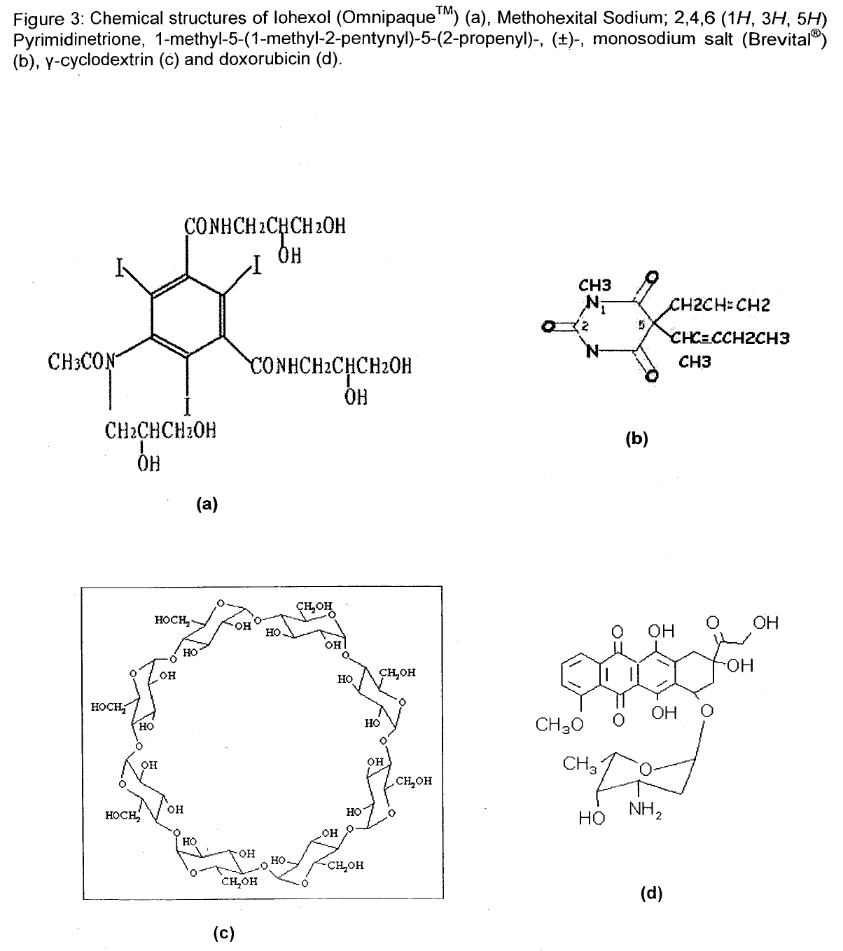

In both examples, after fluorescein infusion, staining was evident on the cortical surface (FIG. 2) and into the deep white matter (FIG. 3). The normal brain tissue autofluoresces slightly at this wavelength. Appropriate filtration on the observation lens can further improve contrast. After infusion of FD & C Green #3, the anterior and middle cerebral artery distributions were clearly stained both on the cortical surface (FIG. 4) and into the deep white matter (FIG. 5), with sparing of the posterior cerebral artery distribution over the inferior portion of the temporal lobe (FIG. 6). Finally, the embolic agent, tantalum powder, produced dark staining of the larger caliber arterial brain cells but was insufficient in its penetration to smaller arterioles and capillaries to be useful as a standalone agent (FIG. 7). While tantalum proved insufficient as a stand-alone agent, this is likely secondary to its size (50-100 mm) and irregular shape (FIG. 8). A more uniform, smaller agent such as Histoacryl® (Braun) added to an oil soluble dye such as Sudan Black B® (Sigma) and Ethiodol (for radioopacity) will obviate the need for disruption of the blood brain barrier, and hasten the procedure.

The toxicity profile of fluorescein or FD & C Green #3 is not severe (Albert et al, supra; Kuroiwa et al, supra; Richard G: Flourescien and ICG Angiography Textbook: An Atlas. Thieme, N.Y., 1998.). Fluorescein is widely used in retinal angiograms, and Fast Green FCF is broadly approved for food and drug use. There is little data on the parenteral use of FD & C Green #3, although there is evidence for mutagenicity in all coal tar dyes (Anonymous: Fast green FCF. IARC monographs on the evaluation of carcinogenic risk of chemicals to man Jan: 16187-97, 1978; Das S K, Gin A K: Chromosomal aberrations induced by secondary and tertiary amine-containing dyes and in combination with nitrite in vivo in mice. Cytobios 54(216):25-9, 1988; Gin A K, Mukherjee A: Sister chromatid exchange induced by secondary and tertiary amine containing dyes and in combination with nitrite in vivo in mice. Cancer-Lett 52(1):33-7, 1990; Hansen W H, Long E L, Davis K J, Nelson A A, Fitzhugh O G: Chronic toxicity of three food colorings: Guinea Green B, Light Green SF Yellowish and Fast Green FCF in rats, dogs and mice. Food and cosmetics toxicology 4(4):389-410, 1966; Ishidate M, Sofuni T, Yoshikawa K, Hayashi M, Nohmi T, Sawada M, Matsuoka A: Primary mutagenicity screening of food additives currently used in Japan. Food and chemical toxicology 22(8):623-36, 1984; Roychoudhury A, Gin A K: Effects of certain food dyes on chromosomes of Allium cepa. Mutat-Res 223(3):313-9, 1989). There is also data to suggest induction of sarcomas after chronic subcutaneous injections of FD & C Green #3 (Nelson A A, Hagan E C: Production of fibrosarcomas in rats at site of subcutaneous injection of various food dyes. Federation Proceedings 12:397-398, 19531953). The clinical impact of any toxicity is expected to be very minimal because of the single exposure, and the ability under fluoroscopy to stop dye infusion once the angiogram shows full perfusion. Any direct toxicity to the stained parenchyma is likely of little clinical significance, as that stain is only applied to tissue that is intended to be removed very shortly thereafter.

Both fluorescein and FD&C Green #3 resulted in good staining of a selective vascular distribution. Tantalum powder darkly marked the main arterial branches but did not show good parenchymal staining. Thus, the invention is highly valuable for the endovascular marking of tissue for clear identification at craniotomy. This is a novel application of endovascular technology with broad potential uses in preoperative brain mapping. For example, as a resection proceeds, the brain tends to shift into the resection cavity and resembles the preoperative scan less and less as the resection proceeds. Thus, it is least accurate at the end of the case, when final decisions as to the limit of resection are most crucial. The brain mapping technique of the invention eliminates such concerns, beginning with minimal invasive clinical testing of the patient. Testing is followed by embolization at the same setting, if necessary, and clear marking of safe resection parameters that will be clearly visible to the neurosurgeon at the time of surgery.

The present invention provides endovascular brain mapping that will define safe margins of resection at craniotomy, as well as any expected neurologic deficit. Selective disruption of the blood brain barrier followed by selective infusion of a dye such as fluorescein or FD & C Green #3 can produce selective staining of the desired vascular distribution as would be expected.

Following are examples of additional strategies for effecting preoperative endovascular brain mapping.

### EXAMPLE 3

### Passive Transport of Dye (Non-invasive Strategy)

Dye molecules (mapping agents) which are sufficiently small (molecular weight less than about 500) can cross the blood-brain-barrier (BBB) and reach brain parenchyma by simple diffusion process. Small lipophilic dye molecules such as fluorescein and 4,4-difluoro-4-bora-3a,4a-diaza-s-indacene (BODIPY), their alkyl derivatives and other similar dye molecules can be used for this purpose. Some representative examples are given below in Table 1.

TABLE 1
Fluorescent compounds (fluorophores) to be investigated as mapping agents.

| Fluorophores | | |
|---|---|---|
| (λ_{exc}/λₑₘ) | Chemical Structure | Solubility |
| 4,4-difluoro-1,3,5,7- tetramethyl-4-bora- 3a,4a-diaza-s-indacene [Tetramethyl- BODIPY ® (505/515)] | 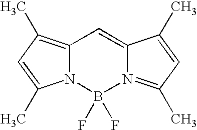 | Cyclodextrins (CD) |
| 5-butyl-4,4-difluoro-4- bora-3a,4a-diaza-s- indacene-3-nonanoic acid [C₄-BODIPY ® (500/510) C₉] | 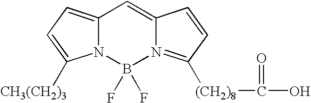 | CDs |
| Fluorescein | 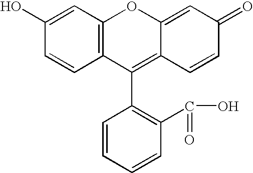 | DDs |
| 5-dodecanoylamino- fluorescein | 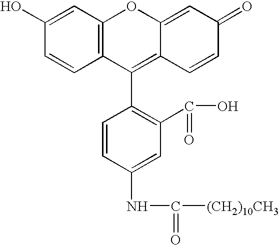 | CDs |

Because of their low solubility in water, some dyes are solubilized in cyclodextrin (CD) in PBS solution. Omnipaque , a radio-opaque contrast agent is mixed thoroughly with the formulation before injection in order to visualize under a fluoroscope. Initially, the formation of an inclusion complex of lypophilic dye derivative and the CD is studied spectroscopically to determine optimal composition. Cyclodextrins (FIG. 3c, Table 2) are sugar molecules having the structure of a hollow truncated cone with a hydrophobic cavity. They (host) are capable of encapsulating appropriately sized fluorophores (guest) in their hydrophobic cavity by forming inclusion complexes. Upon inclusion of a fluorophore, CDs offer a more protective microenvironment and generally enhance the fluorescence of the guest molecule by shielding the excited species from quenching and non-radiative decay processes that occur in bulk solution. In general, the guest molecule loses its solvation sphere upon entering the cyclodextrin cavity, and solvent molecules are simultaneously expelled out from the cavity. Based on the above complex formation mechanism, dye derivatives were selected. The α-CD and unmodified β-CD were excluded because of their small cavity size and low water solubility, respectively (Table 2). 2-hydoxypropyl-β-CD (HP-β-CD, solubility in water: 45% (w/v)), γ-CD (FIG. 3c), 2-hydoxypropyl-γ-CD (HP-γ-CD, solubility in water: 450 mg/mL), carboxymethyl-β-cyclodextrin (CM-β-CD, solubility in water: 50 mg/mL) and carboxyethyl-γ-CD (CE-γ-CD) are suitable candidates for derivative/CD complex formation and they may be lyophilized to produce freely soluble powders. A higher degree of complexation with chemically modified CDs is to be expected due to their high water-solubility. CD solutions at concentrations of 5, 10, 15% (w/v) in saline and differing amounts of dye to be incorporated (working below the saturation level) may be studied to optimize the concentration of CD and/or dye for in vivo brain staining. The dye content of the aqueous phase can be determined by using HPLC. The inclusion complexes can be fully characterized by nuclear-magnetic resonance (H-NMR), UV-VIS and fluorescence spectroscopy and their composition may be optimized to obtain maximum fluorescence intensity.

TABLE 2
Characteristics of α-, β- and γ-CDs.

| | α-CD | β-CD | γ-CD |
|---|---|---|---|
| Number of glucose units | 6 | 7 | 8 |
| Molecular weight | 972 | 1135 | 1297 |
| Solubility in water, g 100 | 14.5 | 1.85 | 23.2 |
| mL⁻¹ at room temperature | | | |
| Cavity diameter, Å | 4.7-5.3 | 6.0-6.5 | 7.5-8.3 |
| Height of torus, Å | 7.9 ± 0.1 | 7.9 ± 0.1 | 7.9 ± 0.1 |
| Approx volume of cavity, | 174 | 263 | 427 |
| Å³ | | | |

### EXAMPLE 4

### “Pro-drug” Approach

Such non-invasive strategies may, for example, entail the bioreversible covalent modification of the dye to improve its physicochemical properties that result in an enhancement of BBB transport.

Some examples are given below.

Active Transport: BODIPY, Fluorescein (as shown above) and other similar fluorescent dye molecules conjugated with nutrients (amino acids, glucose, small peptide vectors etc.) that can be actively transported into the brain. Table 3 shows two representative examples where fluorescein dye is conjugated with peptide vectors.

TABLE 3
Fluorescein [FLUO]-peptide conjugates.

| Fluorophores | Chemical Structure | Solubility |
|---|---|---|
| 5-(aminomethyl) fluorescein, hydrochloride (say FLUO) (to be conjugated with the peptide vector) | 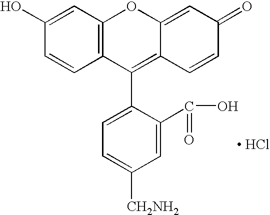 | DMF |
| [FLUO-L-SynB1] | FLUO-RGGRLSYSRRRESTSTGR, 2099 Da | Phosphate |
| | | buffer saline |
| | | (PBS) |
| [FLUO-L-SynB3] | FLUO-RRLSYSRRRF, 1395 Da | PBS |

The pharmacological treatment of brain diseases is often complicated by the inability of potent drugs to pass across the BBB, which is formed by the tight endothelial cell junctions of capillaries within the brain. To overcome the limited access of drugs to the brain different methods have been developed that achieve BBB penetration. Recently, it has been shown that small peptide vectors can be used to enhance brain uptake of therapeutic drugs. These peptide vectors cross cellular membranes efficiently and have been used to enhance penetration of a number of drugs into live cells. The SynB vectors are derived from natural peptides called protegrins. They possess an amphipathic structure in which the positively charged and hydrophobic residues are separated in the sequence. They are able to efficiently cross cell membranes without any cytolytic effect. They have also demonstrated that the mechanism of transport of coupled doxorubicin involves a saturable system, which may operate via an adsorptive-mediated endocytosis. According to the present invention, dyes can be delivered to the brain parenchyma utilizing a similar strategy. The method can be characterized as a “vectorization” of the dyes utilizing the peptide vectors, e.g., vectorization with L-SynB1 (18 amino acids), L-SynB3 (10 amino acids) would significantly increase the brain uptake of fluorescein dye (Table 3).

The peptides may be assembled by conventional solid phase chemistry using a 9-fluorenylmethoxycarbonyl/tertiobutyl protection scheme and purified on preparative C18 reverse phase HPLC after trifluoroacetic acid (TFA) cleavage/deprotection. Purity of the lyophilized products can be assessed by C18 reverse phase analytic HPLC and their molecular weight checked by matrix-assisted laser desorption-ionization (MALDI) time-of-flight mass spectrometry.

FLUO-SynB Synthesis: 5-(aminomethyl)fluorescein hydrochloride (Table 3) is suspended in dimethylformamide (DMF) containing diisopropylethylamine. Succinic anhydride (1 M equivalent) dissolved in DMF is added and incubated for 20 min. The resulting fluorescein hemisuccinate is then be activated by addition of benzotriazol-1-yl-oxopyrrolidinephosphonium hexafluorophosphate (1.1 M equivalent) dissolved in DMF. The peptide is then added to the reaction mixture after 5 min of activation and left for another 20 min for coupling. Purity of the lyophilized products is assessed by C18 reverse phase analytic HPLC and their molecular weight checked by matrix-assisted laser desorption-ionization (MALDI) time-of-flight mass spectrometry.

### EXAMPLE 5

### Receptor-mediated Transport (Receptor-mediated Transcytosis)

BODIPY, Fluorescein (as shown above) and other dye molecules conjugated with transferrin- or insulin-receptor antibodies, immunoliposomes, and the like can be transported into the brain by this transport mechanism.

### EXAMPLE 6

### Adsorptive-mediated Uptake

BODIPY, Fluorescein (as shown above) and other dye molecules conjugated with penetratin, pegellin and the like can undergo adsorptive-mediated uptake process.

### EXAMPLE 7

### Emulsification of Agent

Beta-Catatene emulsification, organic dye emulsification, etc.

### EXAMPLE 8

### Embolization of Brain Capillaries

Described above: (NBCA+Ethiodol+Sudan Black, or Embolization with 5 micron tantalum particles or other dyed particles

### EXAMPLE 9

### Grafted-nanoparticle Systems as Brain-mapping Agents

A variety of dye-loaded nanoparticles/microspheres grafted with the above mentioned “pro-drug” moiety can be used as mapping agents. Fluorescein, BODIPY and similar typs of dyes loaded in biodegradable polymers e.g. polylactic acid (PLA), polylactide-polyglycolide (PLA-PGA) copolymer nanoparticles, n-butylcyanoacrylate (NBCA) nanocapsule-particles can be used as nanoparticulate systems.

Endothelial cells uptake particles grafted with “prodrug” moieties by endocytosis (cellular uptake) process. They can also be strongly adsorbed onto the brain capillaries depending on the surface functionality. The advantages of using particulate systems are:

(i) highly photostable (dye molecules are protected from the environment by polymer matrix,

(ii) Very bright (each particle may contain about tens of thousands of dye molecules). Some of them could be seen by naked eyes.

(iii) More versatile choice of dye molecules (non-toxic and biodegradable organic, inorganic, optical, luminescent and other suitable types including quantum dots (CdS, CdSe, phosphores etc. can be incorporated inside the biodegradable polymer matrix).

(iv) Stable (dyes are incorporated inside the polymer matrix. Therefore, they are stable towards cellular enzymatic decomposition).

It will be understood by those skilled in the art that the method of the invention is equally applicable to any type of surgical procedure involving the brain.

1. A method for diagnostically imaging a preselected region in a warm-blooded animal having a brain situated inside a blood-brain barrier having an ambient permeability level, the method comprising the steps:
endovascularly administering a first chemical agent to the animal at a location proximal to the preselected region outside the blood-brain barrier, where the first chemical agent is capable of increasing the permeability of the blood-brain barrier from the ambient level to an elevated permeability level;
endovascularly administering a second chemical agent to the animal at a location proximal to the preselected region outside the blood-brain barrier following the administration of the first chemical agent, the second chemical agent being capable of staining the preselected region of the brain to a color visibly contrasting with non-stained portions of the brain and of passing through the animal's blood-brain barrier to the preselected region of the brain when the blood-brain barrier is at the elevated permeability level but not at the ambient permeability level.

2. The method of claim I wherein the second chemical agent is non-toxic to the mammal.

3. The method of claim 2 wherein the second chemical agent is a dye.

4. The method of claim 3 wherein the dye is fluorescein, FD & C green #3, [List others ______]

5. The method of claim 1 wherein the second chemical agent also comprises a component that is radiopaque.

6. The method of claim 5 wherein the radiopaque component is [List several ______]

7. The method of claim 1 wherein the first chemical agent is arabinose, [List others ______]

8. The method of claim 1 wherein the mammal is a human.

9. In a method involving surgery on the brain of a warm-blooded mammal, the improvement comprising diagnostically imaging a preselected region in a warm-blooded animal having a brain situated inside a blood-brain barrier having an ambient permeability level, the imaging comprising the steps:
endovascularly administering a first chemical agent to the animal at a location proximal to the preselected region outside the blood-brain barrier, where the first chemical agent is capable of increasing the permeability of the blood-brain barrier from the ambient level to an elevated permeability level;
endovascularly administering a second chemical agent to the animal at a location proximal to the preselected region outside the blood-brain barrier following the administration of the first chemical agent, the second chemical agent being capable of staining the preselected region of the brain to a color visibly contrasting with non-stained portions of the brain and of passing through the animal's blood-brain barrier to the preselected region of the brain when the blood-brain barrier is at the elevated permeability level but not at the ambient permeability level.

10. The method of claim 9 wherein the second chemical agent is non-toxic to the mammal.

11. The method of claim 10 wherein the second chemical agent is a dye.

12. The method of claim 11 wherein the dye is fluorescein, FD & C green #3, [List others ______]

13. The method of claim 9 wherein the second chemical agent also comprises a component that is radiopaque.

14. The method of claim 13 wherein the radiopaque component is [List several ______]

15. The method of claim 9 wherein the first chemical agent is arabinose, [List others ______].

16. The method of claim 9 wherein the mammal is a human.

17. The method of claim 1 or 9 including the step of testing the preselected region of brain for neurological deficit.

18. The method of claim 17 wherein the preselected region of brain is tested for neurological deficit by anesthetization.

19. The method of claim 1 or 9 including the step of resecting the preselected region of brain.

20. The method of claim 1 or 9, including endovascularly isolating the preselected region of brain from the remainder of the brain.

21. The method of claim 20 wherein the preselected region of brain is endovascularly isolated by embolization.

22. The method of claim 21 wherein embolization is enabled by administration of an embolization agent with the second chemical agent.

23. The method of claim 22 wherein the embolization agent is Butyl cyanoacrylate, tantalum [list others ______].

24. A pharmaceutical composition for endovascular brain mapping in a kit form comprising:
a first chemical agent capable of increasing the permeability of the blood-brain barrier from an ambient level to an elevated permeability level, and a second chemical agent capable of staining a preselected region of the brain to a color visibly contrasting with non-stained portions of the brain and of passing through the animal's blood-brain barrier to the preselected region of the brain when the blood-brain barrier is at the elevated permeability level but not at the ambient permeability level.

25. The composition of claim 24 wherein the first chemical agent is arabinose, [List others ______].

26. The composition of claim 24 wherein the second chemical agent is a dye.

27. The composition of claim 26 wherein the dye is fluorescein, FD & C green #3, [List others ______]

28. The composition of claim 24 wherein the second chemical agent also comprises a component that is radiopaque.

29. The composition of claim 28 wherein the radiopaque component is [List several ______].

30. The composition of claim 24 additionally containing an anesthetic.

31. Additional embodiments of the invention relate to the following strategies for endovascularly mapping the brain: 1) Passive transport: (a) pro-drug, (b) Modification of mapping agent to mimic molecules that readily cross the BBB (e.g., amino acid, glucose, etc.); 2) Active transport; 3) Receptor-mediated transport (e.g., antibody mediated); 4) Blood brain barrier (BBB) manipulation; 5) Emulsification of agent (such as β-carotene, organic dye, etc.) to stain capillary endothelium; 6) Embolization of brain capillaries; 7) Grafted-nanoparticle systems for brain delivery of mapping agent.

32. Still further embodiments of the invention relate to kits for endovascularly mapping the brain and their use wherein the kits comprise, as a first component, a chemical agent being capable of staining a preselected region of the brain to a color visibly contrasting with non-stained portions of the brain and, as further components, agents for enhancing transport to the preselected regions of the brain of the chemical agent.

33. Other embodiments of the invention relate to articles of manufacture and their use wherein the comprises packaging material and a pharmaceutical agent contained within the packaging material, wherein the pharmaceutical agent is effective for the endovascular mapping of the brain, and wherein the packaging material comprises a label which indicates that the pharmaceutical agent can be used for the endovascular mapping of the brain.
